(19) United States
(12) Patent Application Publication
(10) Pub. No.: US 2025/0382947 A1
(43) Pub. Date: Dec. 18, 2025

(54) A THERMOACOUSTIC ENGINE DRIVEN BY IRRADIATION OF AN ABSORBING MEDIA OR OSCILLATING HEATING

(71) Applicant: Technion Research & Development Foundation Limited, Haifa (IL)

(72) Inventors: Nathan BLANC, Haifa (IL); Guy Ramon, Pardes Hana Karkur (IL); Yehuda Agnon, Shechania (IL); Avishai Meir, Ashkelon (IL)

(73) Assignee: Technion Research & Development Foundation Limited, Haifa (IL)

(21) Appl. No.: 19/107,547

(22) PCT Filed: Aug. 30, 2023
(86) PCT No.: PCT/IB2023/058586

§ 371 Date: Feb. 27, 2025

Related U.S. Application Data
(60) Provisional application No. 63/373,866, filed on Aug. 30, 2022.

(51) Int. Cl. F03G 7/00; G10K 15/04

(52) U.S. Cl. CPC F03G 7/002; G10K 15/04

(57) ABSTRACT

A thermoacoustic engine that consists essentially of: a heat exchanger; and a resonator that is in communication with the heat exchanger. The resonator includes a media configured to absorb one or more electromagnetic signals. The thermoacoustic engine is configured to receive the one or more electromagnetic signals and generate acoustic power.

### CROSS REFERENCE

This application claims priority from U.S. provisional patent Ser. No. 63/373,866 filing date Aug. 30, 2022, which is incorporated in its entirety.

### BACKGROUND

There is a need, in many applications, for a reliable and efficient heat engines that are cheap and easy to manufacture.

### SUMMARY

There may be provided a thermoacoustic engine and a method for generating energy by a thermoacoustic engine.

### BRIEF DESCRIPTION OF THE DRAWINGS

The embodiments of the disclosure will be understood and appreciated more fully from the following detailed description, taken in conjunction with the drawings in which:

FIGS. 1 and 3A illustrate examples of prior art engines and processes; and

FIGS. 2A, 2B, 3B, 4, 5, 6, 7, 8, 9 and 10 illustrates examples of thermoacoustic engines, and of results related to the thermoacoustic engines.

FIGS. 11-16 illustrate equations related to section “Radiation-Driven Thermoacoustic Energy Conversion in Absorbing Media”;

FIG. 17 illustrates energy conversion mechanisms;

FIG. 18 illustrates an example of temperatures and power;

FIG. 19 illustrates an example of life cycles; and

FIG. 20 illustrates dimensions.

### DETAILED DESCRIPTION OF THE DRAWINGS

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

Because the illustrated embodiments of the present invention may for the most part, be implemented using mechanical components and circuits known to those skilled in the art, details will not be explained in any greater extent than that considered necessary as illustrated above, for the understanding and appreciation of the underlying concepts of the present invention and in order not to obfuscate or distract from the teachings of the present invention.

Any reference in the specification to a thermoacoustic engine should be applied mutatis mutandis to a method for using the thermoacoustic engine.

Any combination of any module or unit listed in any of the figures, any part of the specification and/or any claims may be provided.

Thermoacoustic engines are relatively a new method of energy conversion, converting heat into acoustic power by thermodynamic processes occurring inside the sound waves. In the process, heat is transferred from a hot reservoir to an ambient one.

The generated sound wave is a form of mechanical power can be used for heating, cooling, electricity generation and other uses

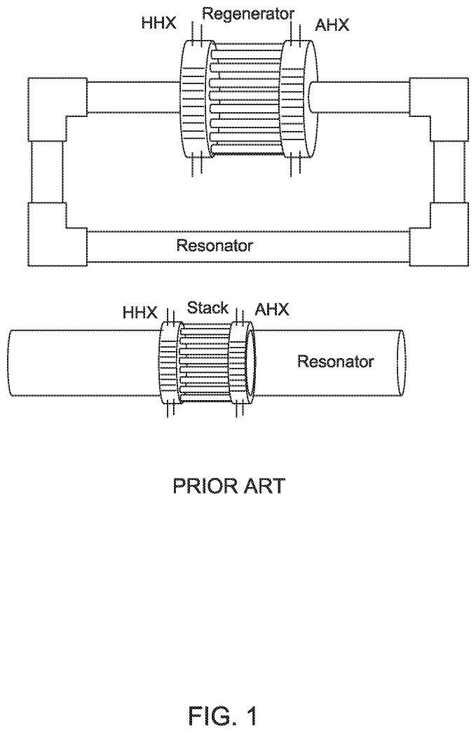

The heart of these engines is a porous component known as a “Stack” or “regenerator”, sandwiched between a hot exchanger (HHX) and an ambient heat exchanger (AHX), each in contact with the corresponding reservoir. See, for examples of a standing wave thermoacoustic engine and of a traveling wave thermoacoustic engine in FIG. 1.

The porous part and two heat exchangers are properly placed in an acoustic resonator, that determines the frequency and from of the sound wave. A straight resonator imposes a standing wave, and a tortuous one can impose a travelling wave.

The pore size is matched with the thermal penetration depth

$${δ}_{α} = \sqrt{\frac{α}{2 ω}} .$$

Travelling wave engines require a pore size much smaller than δ_{α}, while standing wave engines require a pore size slightly larger than δ_{α}

Both standing and travelling wave engines are limited in efficiency. Standing Wave engines have an inherent thermodynamic irreversibility, limiting practical devices to less than 20% of Carnot's efficiency. Travelling wave engines are inherently reversible, but the small pore size leads to substantial viscous losses, limiting the maximal efficiency of existing devices to around 40% of Carnot's efficiency.

Along with the viscous losses (which exist in standing wave devices as well) the stack also causes parasitic heat fluxes due to conduction that further impede performance.

There is provided a thermoacoustic engine, where the porous part and the hot heat exchanger are removed from the system.

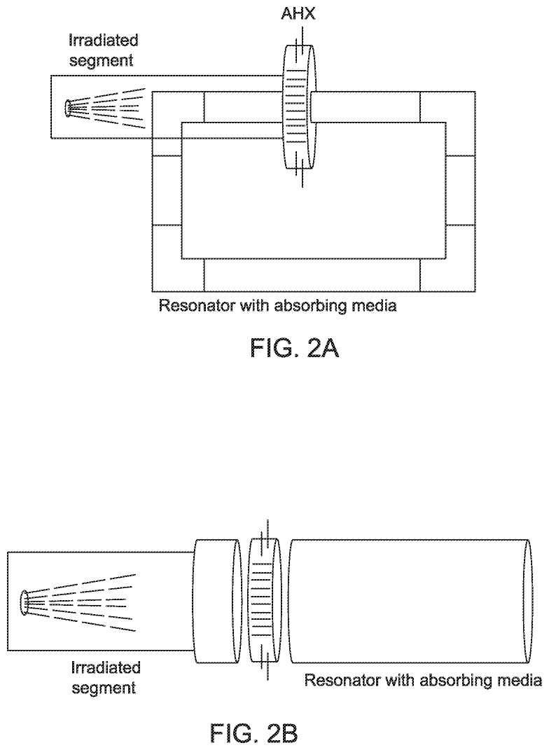

FIGS. 2A and 2B illustrate examples of a travelling wave thermoacoustic engine (FIG. 2A) and standing wave thermoacoustic engine (FIG. 2B). In both cases the thermoacoustic engine includes a AHX and a resonator that is filled with an optically absorbing (and therefore also emitting, due to Kelvin's theorem) fluid, which is irradiated by a radiation source near the ambient heat exchanger.

A temperature gradient is induced by absorption and reemission of radiation. The fluid has to be compressible, and properly absorbing in the wavelength of the radiation source, the radiation source could be concentrated solar radiation, or any other source of concentrated radiation such as photoluminescence, Laser, combustion or other high temperature processes.

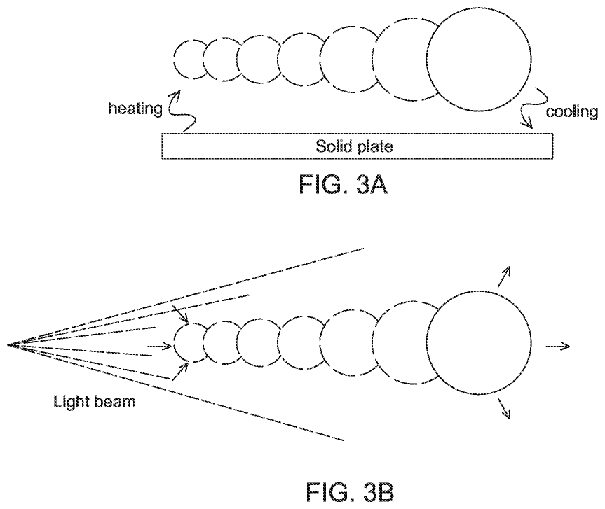

Inside a radiative field, an oscillating gas can go through a thermoacoustic process similar to the one occurring in classical thermoacoustic engines. FIG. 3A illustrates a prior art propagation while FIG. 3B illustrates the propagation within the suggested thermoacoustic engine.

In a sound wave a parcel oscillates spatially, while periodically compressing and expanding. Inside the engine, it interacts with the non-uniform radiative field, absorbing net radiative heat while compressed, and emitting net radiation while expanded. This leads to net acoustic power generation during the acoustic period, converting the radiated heat to acoustic power.

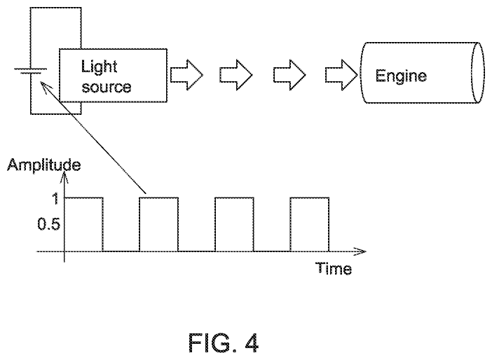

FIG. 4 illustrates an example of a periodic and non-continuous illumination that may trigger a photoacoustic excitation of an absorbing media, either an absorbing fluid or a solid in contact with a fluid. If either of these media is irradiated periodically, acoustic waves are spontaneously generated. This phenomenon is known from various fields such as photoacoustic tomography and photoacoustic spectroscopy. However, it has not yet been used in the field of thermoacoustic energy conversion. We suggest that it could significantly improve the performance of thermoacoustic engine. The source of the oscillating radiation could be a laser or electrically generated light which is periodically switched on and off, (FIG. 4).

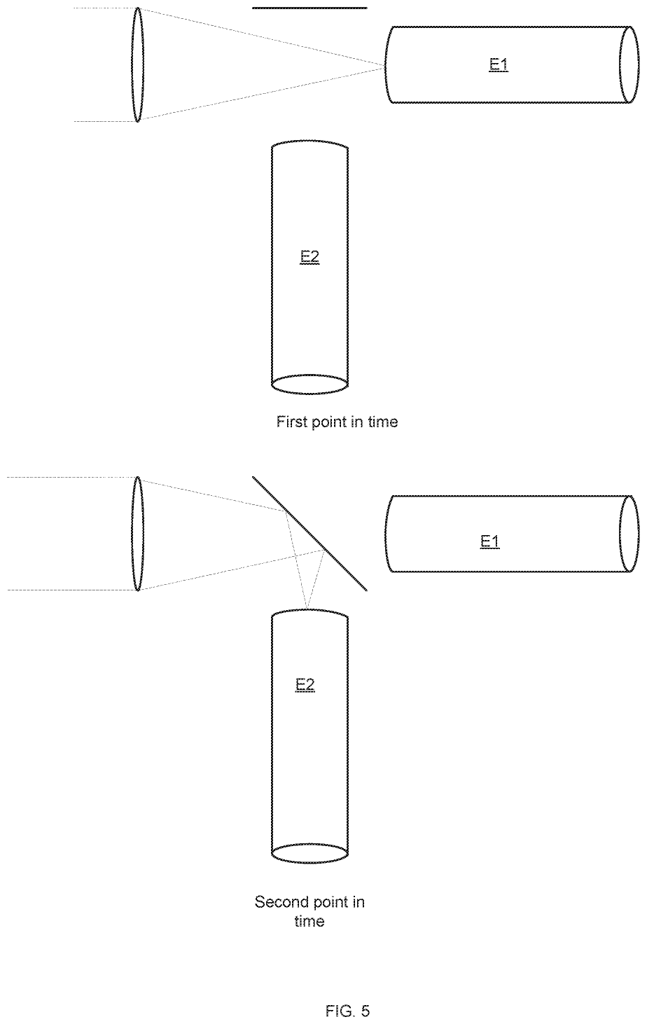

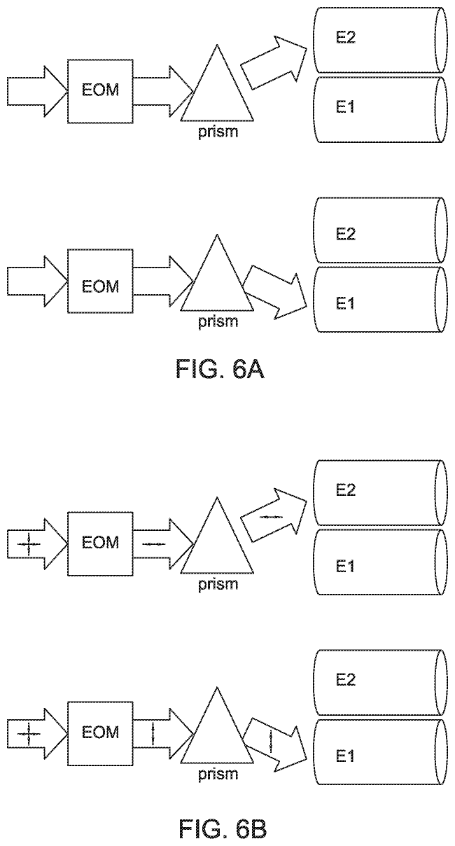

Alternatively, it could be any source of light that is periodically directed towards and away from the absorbing media. For example, a concentrated solar beam can be directed periodically between two thermoacoustic engines, and thus serve as an oscillating radiation source for both of them at once. (see FIGS. 5 and 6).

The spatial manipulation of the light could be via a rotating mirror (FIG. 5), or by combining an Electro-optic modulator (EOM) with a suitable prism. The EOM is used to periodically modify the wavelength (FIG. 6A) or the polarization (in FIG. 6B) of the incoming radiation. The Prism is used to spatially separate the radiation leaving the EOM, directing it to E1 or E2 alternately.

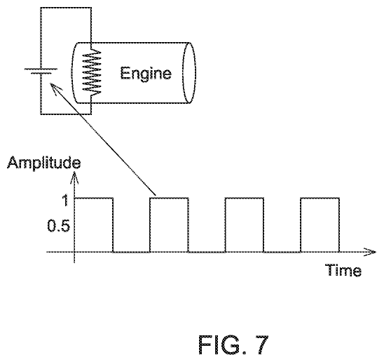

FIG. 7 illustrates an example of a thermoacoustic engine that is not dependent on radiation, and instead utilizes a periodically interrupted current through an electric resistor. This periodical heating of the resistor leads to a very similar effect to photo acoustics, and if placed in a thermoacoustic engine can drive powerful acoustic waves.

The thermoacoustic engine may be used for various purposes. For example—the thermoacoustic engine may be used for solar energy conversion, making it a cheap, reliable and possibly efficient engine, in addition it can be used to generate acoustic power form various radiation sources, form electric power, and from any high temperature source where gases emit radiation. The produced acoustic power can be used for cooling, heating water pumping, electricity generation and propulsion.

The thermoacoustic engine replaces the traditional “heart” of thermoacoustic engines—the porous part, with a radiative field or an oscillating heat source-which imposes severe limitations on the energy conversion rate.

A group of thermoacoustic engine that receive pulses of radiation—may increase the energy conversion rate-especially when no input energy is waisted to provide gaps between the radiation.

The thermoacoustic engine is highly efficient, as it removes a main source of losses (the major one in travelling wave engines) from thermoacoustic engines.

The thermoacoustic engine removes additional parasitic losses, mainly ones due to solid conduction in the porous part.

### Powerful Photoacoustic Oscillations Driven by Periodic Interface Irradiation

Thermoacoustic refers to the interactions of the oscillating Pressure, velocity and temperature fields in acoustic waves. This interaction can lead to energy conversion between heat and acoustic power. Thermoacoustic engines (converting heat to acoustic power) and heat pumps (Converting acoustic power to Heat), have been utilized in various applications, from electricity generators for developing communities to space applications. Thermoacoustic energy conversion is always based on proper phasing of the oscillating temperature and pressure fields p1, T1. Usually, This phasing is achieved by spatial gradients in the mean temperature profile. However, It has been Known for years that periodic irradiation of an absorbing media can lead to a similar effect, known as the Photo-acoustic effect.

Bell was the first to notice and describe this effect. Since then it has been applied in Bio-medical imaging, Spectroscopy, and other uses, but never considered for energy conversion. The introduction of phase change into the acoustic cycle in Thermoacoustic systems has been shown to improve performance substantially, due to the addition of latent heat to the sensible heat transferred during an acoustic cycle. this has been shown both experimentally and theoretically. The effect of phase change on photo-acoustic conversion was also discussed, mostly in relation to photoacoustic interaction of aerosols.

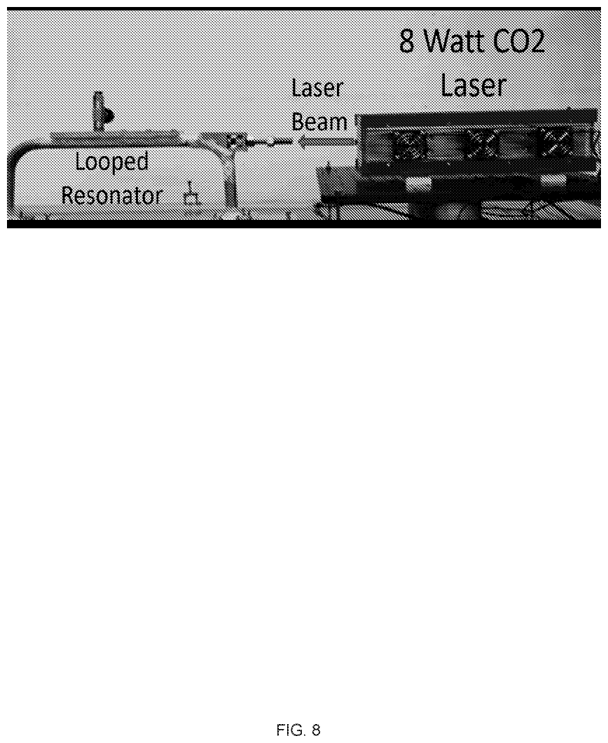

In this work, we report experimental and numerical demonstration of powerful photoacoustic oscillations, driven by periodic irradiation of a looped resonator. We enhance these oscillations by wetting the resonator and adding phase change to the acoustic cycle. The Experimental system is presented in FIG. 8.

An 8 Watt CO2 (MERIT-S Stabilized CO2 Laser by Access Lasers) was directed into a 2.67 m looped resonator. The resonator consisted of a 16 mm diameter, 0.67 m length Aluminum pipe, connected through conic sections to a 21 mm diameter, 2 m length Steel pipe. The laser beam entered the resonator through an 8 mm hole in one side of Aluminum section, and hit the opposing end.

Periodic Ir radiation was achieved by introducing a periodic square wave signal to the laser input source, with different frequencies.

In some experiments, labeled “wet” experiments the resonator was wetted with water by passing a wet rag through the aluminum section three times. The pressure was measured using a pressure sensor, placed 1.2m away from the location of the hole in the resonator.

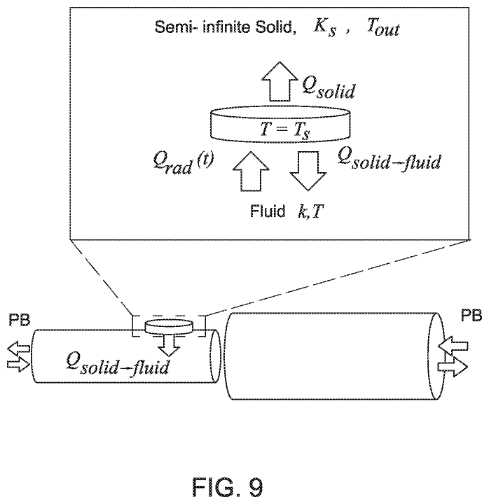

A Numerical analysis was performed for a reduced order model of the system, accounting for the conduction in the solid, the radiation, and the fluid dynamics. Model is schematically presented in FIG. 9.

The Irradiated region of the resonator wall was modelled as a circle of the beam diameter Dbeam, in thermal contact with the laser beam, with a semi-infinite solid on one side and a fluid on the other. The heat transfer from the laser was assumed to be of the form:

$${Q}_{ra d} ( t ) = {Q}_{ma x} \frac{1 + sin ( ω t )}{2} , \qquad ( 1 )$$

Where Qmax=8 [W] is the rated laser power and ω is the angular frequency. The heat transfer to the infinite solid to the solid was calculated based on [15]:

$${Q}_{solid} = {Sk}_{s} ( {T}_{s} - {T}_{out} ) , \qquad ( 2 )$$

Where S=2Dbeam is the shape factor, kS is the thermal conductivity of the solid and Tout=300 [k] is the room temperature.

The Heat transfer between the solid and fluid was calculated based on

$${Q}_{solid -> fluid} = ψ ( {T}_{s} - {T}_{f} ) + \dot{m} {l}_{h} \qquad ( 3 )$$

Where ψ is the heat transfer coefficient, to be discussed in the next paragraph, lh is the latent heat of water, and m\dot{ } is the mass flux of water through evaporation and condensation, given by:

$$\dot{m} = \{ \begin{matrix}0 & dry \\ \frac{ψ}{{c}_{p} Le} ( {e}^{\frac{{l}_{h} ( γ - 1 ) ( {T}_{b} - {T}_{s} )}{{c}_{p} γ {T}_{b} {T}_{s}}} - {e}^{\frac{{l}_{h} ( γ - 1 ) ( {T}_{b} - {T}_{f} )}{{c}_{p} γ {T}_{b} {T}_{f}}} ) & wet\end{matrix} \text{?} \qquad ( 4 )$$

$$\text{?} \text{indicates text missing or illegible when filed}$$

Where cp is the heat capacity of the fluid, Le=α/D is the Lewis Number for an air-water mixture, Tb is the boiling point of water, and γ is the specific heat ratio. The above equations were solved together with the transient, 1D compressible fluid equations (The continuity equation, the momentum equation and the energy equation) with

$$\frac{{Q}_{solid -> fluid}}{π {r}^{2} {D}_{beam}} \qquad ( 5 )$$

as a source term in the energy equation. An accurate value of Y proved difficult to obtain from literature. While many correlations for heat transfer in oscillating flow exist, most of them were calculated for much larger acoustic displacement fields or much smaller pipe sizes and account for the cycle averaged heat transfer coefficient or time averaged Nusselt number, rather than the temporal one. More importantly, these correlations are for a purely parallel flow. While in most of the resonator the flow is indeed purely parallel, in the vicinity of the irradiated point in the resonator, Perpendicular flow is to be expected as well. The temperature oscillations and their coupled pressure oscillations are expected to drive air to and away from the irradiated wall. The magnitude of the heat transfer due to these oscillations is difficult to evaluate, but it can be expected to be proportional to both the acoustic displacement ζ=v1/ω and to the thermal penetration depth δα=square root of (α/2ω), Where v1 is the acoustic velocity amplitude in the radial direction amplitude, and a is the thermal diffusivity.

Therefore, we chose to model ψ as:

$$ψ = \frac{{c}_{1}}{{ω}^{1.5}} \qquad ( 6 )$$

Where c1 is a proportionality constant to be determined experimentally. The optimal value of c1, where agreement with experimental data was maximal, was:

$${c}_{1} ( optimal ) = 1.65 \cdot {10}^{5} [ \frac{{Js}^{0.5}}{k} ] \qquad ( 7 )$$

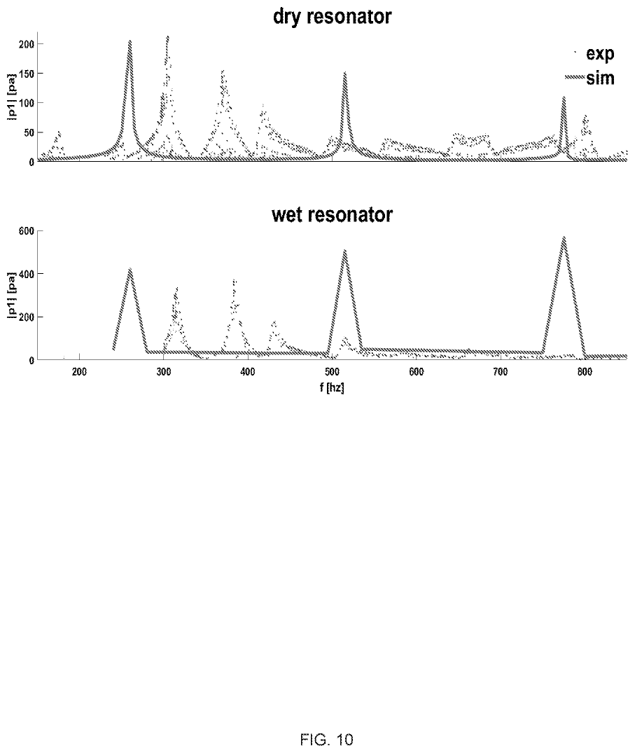

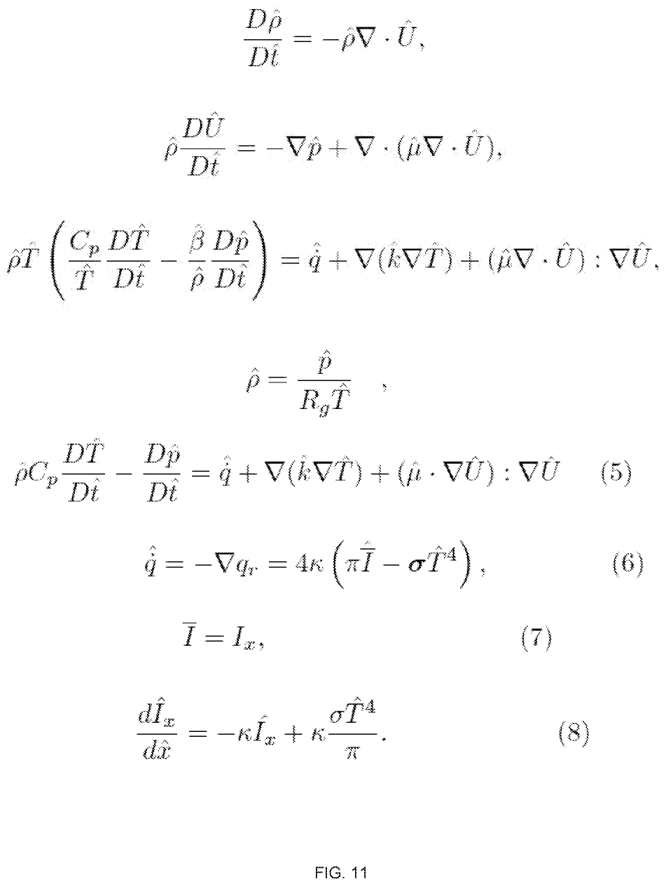

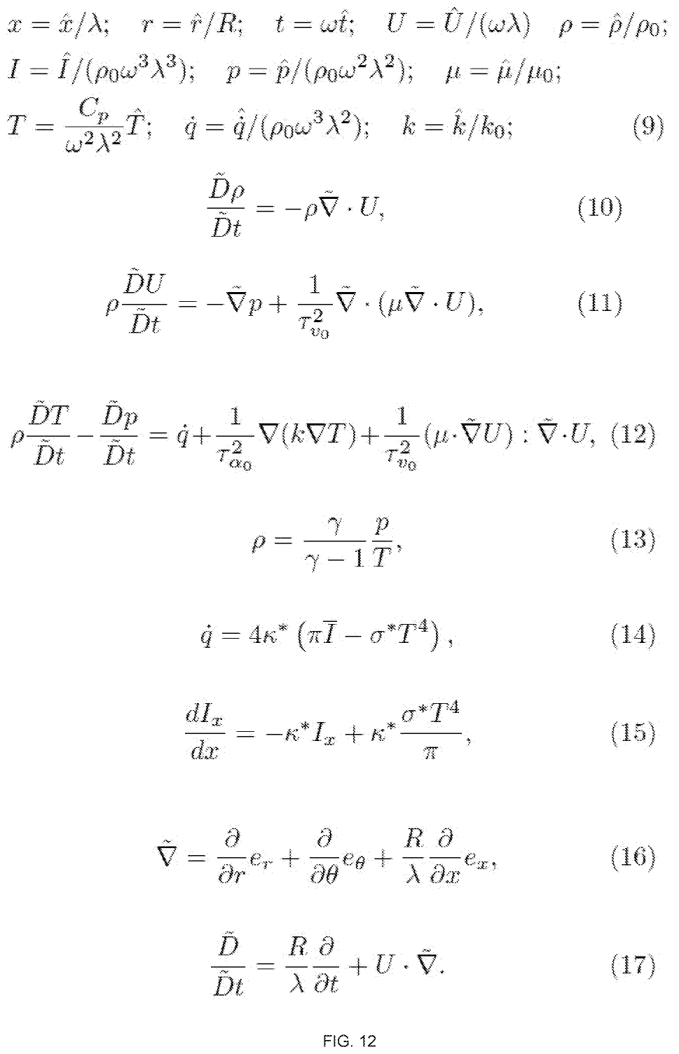

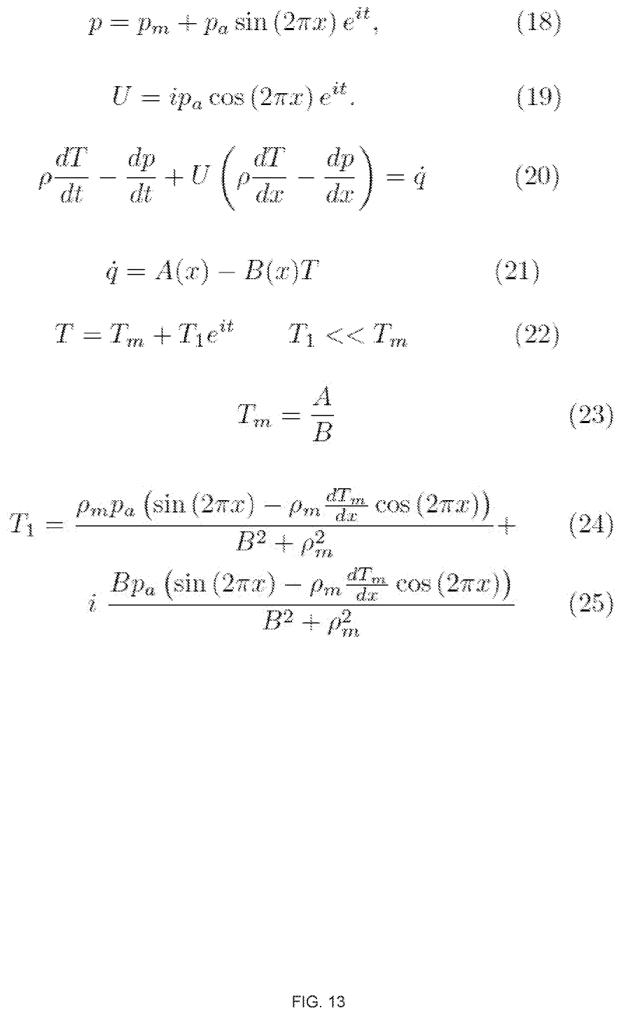

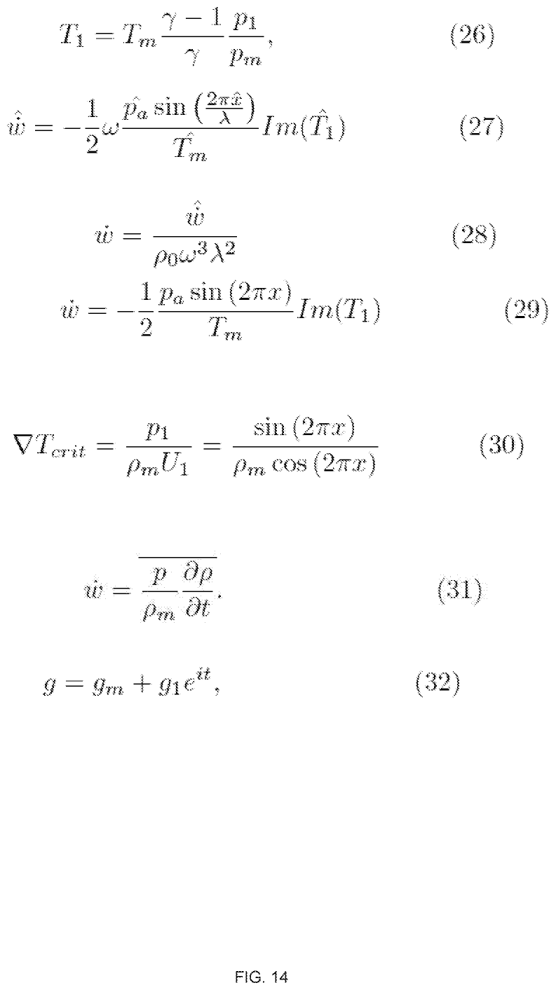

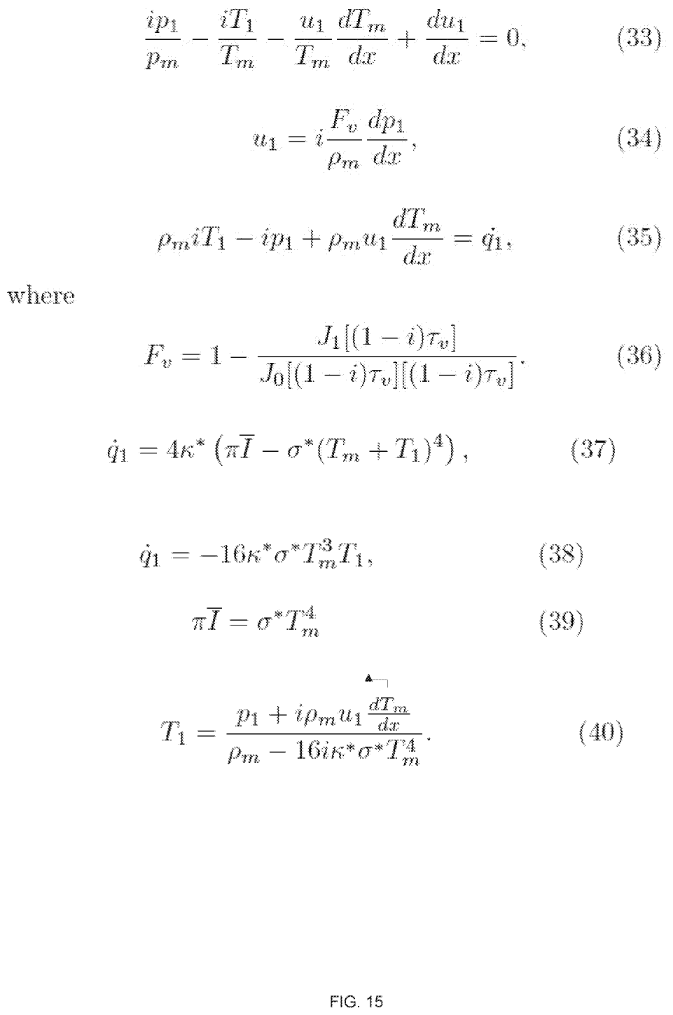

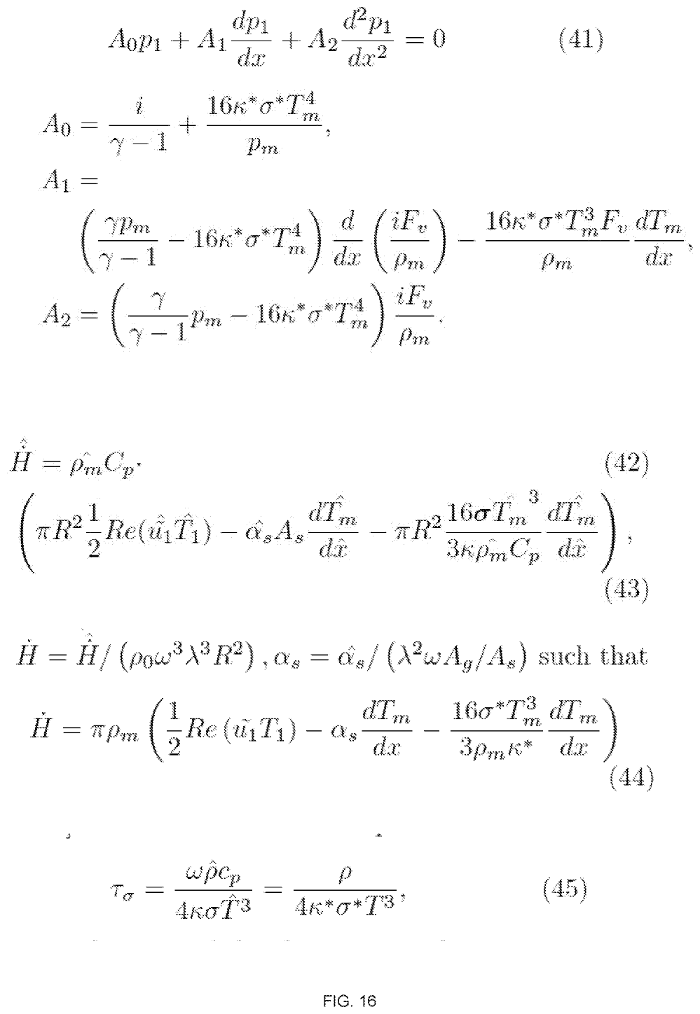

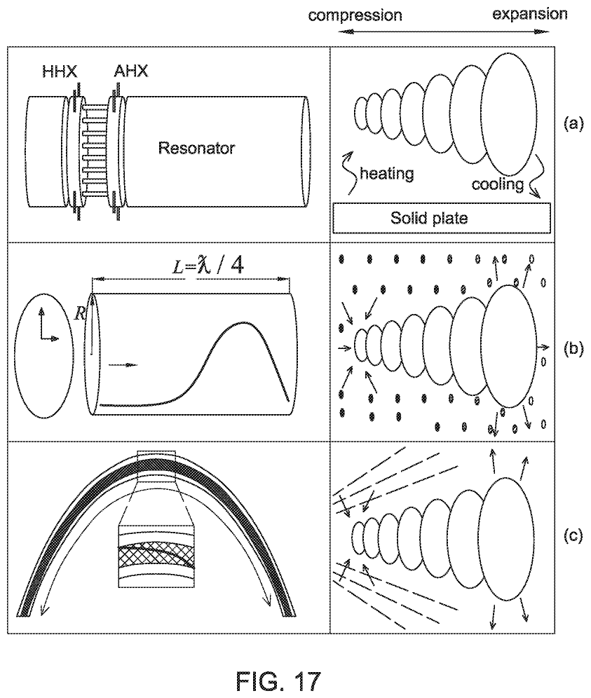

The equations were solved using the upwind method, using the Crank-Nicholson method for temporal integration and a periodic boundary condition on both ends of the domain. The time steps and grid sizes were Δt=2 [μs] and Δx=2.22 [mm] respectively. Time integration continued until a limit cycle was reached and the amplitude was defined as the difference between minimal and maximal pressure in the cycle. Numeric and Experimental results for both modes are presented in FIG. 10, displaying the maximal pressure amplitude as a function of the driving frequency of the laser.

Experimental results indicate a pressure amplitude of up to 250 [Pa] (141 [DB]) for the dry mode, and up to 380 [Pa] (145 DB) for the wet mode. According to Swift, this corresponds to a Dissipated acoustic power of about 0.02 [W] and 0.06 [W], respectively. If the temperature profile from the model is used to calculate the temperature differences, This corresponds to conversion efficiencies of about 30% and 90% of Carnot's efficiency.

Notably, the introduction of phase change into the acoustic cycle more than doubles the conversion efficiency and the generated acoustic power. An interesting trend in both numeric and experimental results, is the non-monotonous trend in amplitudes with the frequency.

In the wet mode, the highest amplitude is not even at the fundamental frequency, but at the first harmonic. This counter intuitive result could be explained by the positive dependence of thermoacoustic conversion efficiency on frequency, as described by Swift. However, this improved efficiency is countered by enhanced viscous losses and thermal relaxation at higher frequencies, as well as the inverse relation between ψ and ω. The trade-off between these effects leads to an optimal working frequency that is not necessarily the fundamental frequency.

There is some qualitative agreement between the simulations and experiments, and pressure amplitude. In addition main trends discussed previously appear in both calculations and experiments. Two important deviations should be noted. First, while the theory predicts resonance occurring at the “text-book” resonance frequencies (fn=na/L, where n is an integer), the actual resonant frequencies were very different. This

Deviation could be attributed to hole in the resonator through which the radiation entered it. Such holes are known to have significant influence on both the resonant frequencies and their corresponding sound pressure levels. Letting The radiation in through a window could yield better agreement with theory. Alternately, the hole could be introduced into the model by calculating its acoustic impedance. Another significant deviation occurs at the wet system with higher frequencies. The disagreement between the numeric and experimental results there is substantial, and spans orders of magnitudes. Contrary to the lower frequencies, At these frequencies the performance of the wet resonator was much poorer than that of the dry resonator. This could be related to some form of mass dispersion or mass streaming that was not captured by the simplified model presented here, to the accumulation of water at some point in the resonator, or to variations in the value of ψ. We note that the assumption of a constant value for ψ is inherently erroneous, as in reality the thermal contact varies during the acoustic cycle.

The results presented here are a first step towards designing a new type of thermoacoustic engines, based on alternating radiation. While alternating radiation at such frequencies is of not a naturally occurring phenomena, a resonator could be periodically illuminated by periodic intermission of a light source, or by alternately turning the radiation source towards and away from the resonator. If the efficiency of such devices could be improved, they could be used to convert solar energy into sound waves, which in turn could be used for electricity generation, cooling and additional purposes. Extensive research is required to investigate the performance of such devices at higher pressures and higher heat fluxes corresponding with higher energy densities. This performance can be investigated and optimized both experimentally and theoretically. The theoretical framework should be extended to include both a full scale computational model, to bridge the gap between theory and experiment, and a simplified linear model allowing for quick, albeit imprecise performance evaluations.

### Radiation-Driven Thermoacoustic Energy Conversion in Absorbing Media

The equations in this section are numbered (1) till (45).

Thermoacoustic engines rely on an instability that can convert heat into an intense acoustic field-oscillating pressure and velocity. Being heat-driven, they are a promising option for solar energy harvesting and, moreover, have the potential to be cheap, reliable, and robust heat engines, with no moving parts and no exotic materials. Herein we present an as-yet overlooked mode of triggering a thermoacoustic instability in such engines, driven by radiation. This mode of operation can potentially eliminate most of the viscous losses associated with high amplitude thermoacoustic energy conversion. A theoretical framework is developed for modelling such devices, with a simplified analytical analysis illustrating that any non-uniform, temperature dependent heat source has the potential to drive an acoustic instability. It is further demonstrated that radiation can indeed drive such an instability, and the stability limit is identified for different configurations. Results show that the instability is triggered more easily when the oscillation period is matched with the characteristic radiative heating time of the gas. A limit cycle analysis indicates potential efficiencies of up to 90% of Carnot's efficiency, and power densities of up to 5 MW/m³. While several assumptions made may result in lower performance for a real system, the model demonstrates that this approach offers great potential for future development.

The term thermoacoustic refers, in general, to thermal interactions in sound waves. A particular form of thermoacoustic interaction occurs when a fluid is in contact with a solid media, and a temperature gradient is imposed, large enough to trigger an acoustic instability, resulting in spontaneous generation of intense sound waves. The instability is facilitated by appropriate phasing of heat transfer and pressure variations in the fluid.

Essentially, if the gas is heated when compressed and cooled when expanded, any acoustic disturbance will be amplified, in complete analogy with the thermodynamic cycle of a heat engine. This mechanism allows for conversion of heat to acoustic power, a form of mechanical power, which can then be used for electricity generation, cooling and additional applications. Thermoacoustic engines have been considered for cases where the heat is supplied by combustion, industrial waste as well as solar collectors), and offer the advantage of cheap manufacturing, reliability, and high energy density. Early research on thermoacoustics was documented, demonstrating the generation of sound via heating. Rott's theory was further developed and designed several working thermoacoustic engines and refrigerators. These engines rely on heat conduction between a gas and solid “stack”—an array of pores used to increase the contact area of the gas with the solid).

The motivation for research on thermoacoustic energy conversion is evident. Over the past decade, global energy consumption has increased by more than 30 percent and is expected to further increase by more than 50 percent by 2050.

Current energy production is dominated by fossil-fuel-based methods, and therefore poses a threat to the environment. While several sustainable energy production methods exist, many of them (e.g. hydroelectric, geothermal) are limited in scope to specific geographic conditions, or in other ways unsuited for Global scale energy production. Solar power stands out as large scale, globally distributed source, and is considered to be the most likely candidate to replace fossil fuels]. Current common solar energy production methods include photovoltaic cells and large scale thermal energy power plants. The latter is relatively expensive, and still not competitive with conventional technology, while the former is limited in efficiency, and therefore also in energy produced per unit area. The stack has been an essential part of most known thermoacoustic devices and is usually referred to as the ‘heart’ of the device. Early thermoacoustic engines supported a standing wave acoustic field, where the pressure and velocity oscillations are almost completely out of phase. A standing wave device requires stack pore sizes on the order of the thermal penetration depth, 8=p a/@, where a is the thermal diffusivity of the gas and @ is the acoustic frequency.

This leads to entropy generation due to imperfect thermal contact, but is required for maintaining the exchange of heat necessary for amplifying and sustaining the oscillations. The inherent irreversibility substantially limits the efficiency of standing wave engines, with maximal efficiencies limited to 20% of Carnot's efficiency.

There has been suggested an implementation of travelling wave acoustic fields, which have the potential for higher efficiency due to the reversible nature of the heat transfer occurring at near isothermal conditions. However, accomplishing this requires excellent thermal contact, which dictates that the pore size be much smaller than the thermal penetration depth. While perfect thermal contact reduces entropy generation, allowing for efficiencies up to 40% of the Carnot efficiency and power outputs of 5 [kw], smaller pores lead to substantial viscous losses.

This situation is inherent to almost any conduction driven thermoacoustic engine. A reversible thermodynamic cycle requires perfect thermal contact, but perfect thermal contact means good viscous contact, for any fluid with a moderate Prandtl number (as most compressible fluids have). Therefore, it is of interest to investigate alternative modes of energy transfer in thermoacoustic devices, where losses can be further reduced.

An alternative mode also considered is heat transfer coupled with mass transfer due to phase change, absorption or adsorption. There has been suggested incorporating mass transfer into thermoacoustic engines, and more recent studies demonstrated the superior performance of phase change engines over conduction driven ones. While these engines involved modes of heat transfer other than conduction, they still required a solid interface for operation, and therefore suffer from the same problem of enhanced viscous losses in travelling wave devices.

A clue pointing at the fact that a solid interface is not an essential requirement for thermoacoustic engines can be found in a slightly different field—the suppression of thermoacoustic instabilities in internal combustion engines and gas turbines. This is an intensively researched field, where the heat input is often volumetric, or at least modelled as such. Indeed, in numerical investigation of thermoacoustic engines, especially for travelling devices, the stack is often replaced by a heat generation term in order to simplify calculations, further demonstrating that volumetric heat generation can cause thermoacoustic effects similar to those created by a solid-fluid interface. The generation of acoustic waves using radiation is used in the bio-medical field, for photoacoustic tomography (PAT), where these waves are used for high-resolution multi-scale imaging of body tissues.

This demonstrates that radiation can indeed be used to drive acoustic waves.

However, the acoustic propagation in PAT is in a soft solid medium, rather than a gas, and the acoustic power output is negligible. There has been experimentally demonstrated a radiation driven thermoacoustic phenomena, providing further evidence to the existence of such a mechanism. The present study is aimed at theoretically investigating the characteristics of radiation-driven thermoacoustic instabilities in the context of thermoacoustic engines, with particular emphasis on the conditions required for their occurrence, and their implications for energy conversion.

### Governing Equations and Non-Dimensionalization

Consider a Newtonian fluid contained within a cylindrical, quarter-wavelength acoustic resonator. The resonator has a radius R and length λ/4>>R, where λ is the acoustic wavelength and a cylindrical coordinate system is employed (\hat{ }x, \hat{ }r, \hat{ }θ).

For initial simplicity, we introduce a general volumetric heat source \hat{ }q\dot{ } (x\hat{ }, T\hat{ }), to represent the expected effect of radiation-induced heating. The governing equations are the mass, momentum and energy conservation equations: see equations (1)-(3)

Where \hat{ }p is the density, \hat{ }U the velocity vector, \hat{ }p the pressure, \hat{ }T the temperature and \hat{ }t the time. \hat{ }u is the viscosity and k\hat{ } is the thermal conductivity. β\hat{ } and C\hat{ }p are the thermal expansion coefficient and the heat capacity, respectively. We also assume an ideal gas equation of state, as shown in equation (4) in which Rg is the substance's gas constant, i.e the universal gas constant divided by the molar mass. With the additional assumptions of a calorically perfect gas (Cp=const, \hat{ }β=1/\hat{ }T) equation 3 is reduced to (see equation (5)).

In an irradiated system, under the assumption of a grey, non-scattering medium, the radiative heat can be written as a volumetric heat source, (see equation (6)). Where qr is the radiative heat flux, K is the absorption constant, \hat{ }I is the radiation intensity averaged over all wavelengths and σ is the Stefan-Boltzmann constant. The mean radiation intensity, \hat{ }I, should generally be obtained by solving the radiative transfer equation (RTE) in different directions. However, if the domain is perfectly insulated from radiation and heat transfer in the radial direction, the mean intensity would be the intensity in the axial direction, (see equation (7)). and the RTE in the axial direction is given as (see equation (8)). Equations (6-8) provide the full model for radiative heat transfer within the framework considered in this paper. Next, we non-dimensionalise the equations, using the following substitutions: (see equation (9)).

Where ω=2πa/λ is the angular frequency, with a denoting the speed of sound. Subscripts ‘0’ denote reference values for different quantities, and the that denotes a dimensional quantity. The non-dimensional form of equations (1-8) is then (see equations (10-17)). Here, ei is the unit vector in direction i, τv0=R p ω/v0 and τα0=R p ω/α0 represent the scaled conduction and viscous times. γ is the specific heat ratio. κ*=κλ and σ*=σω⁵λ⁵/(ρ0C4 p) are the scaled absorption coefficient and Stephan-Boltzmann constant, respectively.

### Simplified Model

We begin by investigating a simplified model of a radiation-driven thermoacoustic wave. The purpose of this model is to gain qualitative insight into the thermoacoustic energy conversion mechanism, by neglecting all but the core characteristics of the flow field. We neglect effects such as viscous stresses, thermal conductivity, all two dimensional effects, and conduction. We ignore the radiative field and the radiative transfer equation, and simply assume a given volumetric heat source q\dot{ }(x, T).

Most importantly, the effect of irradiation on the wave itself is neglected, which means that the density in equations 10 and 11 is dependent only on the pressure.

This assumption is valid only for very small temperature variations.

However, it is very useful because it decouples equation 12 from equations (10-11), permitting an analytical solution.

Under the assumption of small oscillations, we solve equations (10-11) with a no penetration boundary condition at x=¼ and with a free surface boundary condition at x=0. The solution is a simple, ideal acoustic standing wave, in the domain 0<x<¼ (see equations (18-19)). And equation (12) turns to equation (20).

We introduce a volumetric heat source, of the form of equation 21. Where A and B are some functions of X, and T is the temperature. The addition of a temperature dependent term to the heat source is at the heart of the proposed thermoacoustic mechanism. The dependency was assumed linear in this section in order to simplify the analysis.

We now proceed by assuming that the temperature, like the pressure and velocity, takes the form: of equation (22). We divide the energy equation into zeroth and first order equation, and solve for Tm and T1 respectively. The solutions are: equations (23-15).

Several things may be pointed out using this solution. First, in the absence of a heat source, the sound wave is adiabatic and the solution is reduced to the known oscillatory temperature in adiabatic sound waves (see equation (26)). Where p1 is the oscillatory pressure. We note that equation 23 is not valid in the adiabatic case, and any constant value of Tm is valid solution in this case. Importantly, we see that the presence of a heat source leads to an imaginary part of T1, meaning that the temperature is no longer in phase with the pressure. This is important since, as shown by, the existence of an imaginary part of T1 leads to absorbed or generated acoustic power per unit volume, which can be estimated as indicated in equation (27).

We scale the power based on equation (28) and obtain equation (29).

Finally, we notice that there is a critical temperature gradient in our solution, where the acoustic power turns from negative to positive—as illustrated in equation (30).

This critical gradient is identical to the critical temperature gradient presented by. However, in our case this is a result of a volumetric heat source, rather than interaction with a solid interface.

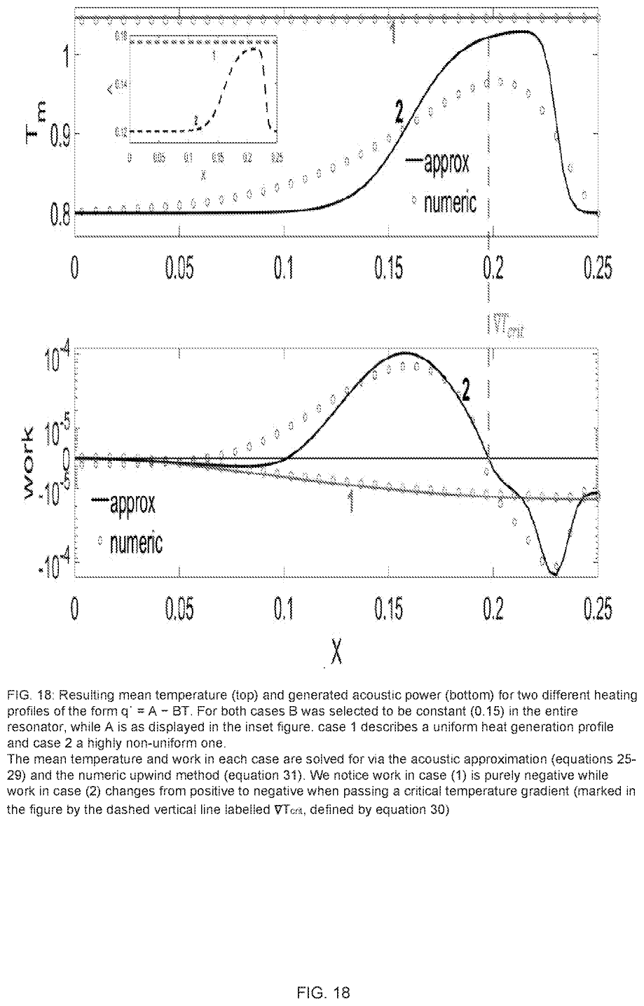

Next, we examine two examples of solutions for different heating profiles, characterized by A(x) and B(x). The two heating profiles represent a uniform heat generation profile and intense heating at a specific location inside the resonator, as can be seen in the inset of FIG. 18. For both cases the mean pressure was set to unity, the scaled pressure amplitude is set to 0.05, and the specific heat ratio is 1.4. In order to validate the simplified, linear model, equation 20 was also solved directly, using an upwind scheme. The time integration was performed using a two stage Runge-Kutta method, with the discretization steps for t and x taken as 1200 and 1300, respectively.

The solution was time-averaged to evaluate the mean temperature, and the work was evaluated (again following) as equation (31). The calculations of the mean temperature profile are presented in FIG. 18, demonstrating good agreement between the linear and non-linear models, with a slight deviation in case 2, especially in the locations where the linear model predicts strong gradients. The deviation may be attributed to higher order effects, and specifically acoustic streaming, which is known to be stronger in the presence of sharp gradients. The agreement is quite good because we are in the limit of small amplitudes (p1/pm<<1), making non-linearities inherently weak.

The calculated acoustic work is presented in the bottom of FIG. 18. In the first case (uniform heat generation) the acoustic power is negative within the entire domain.

This is similar to the introduction of an isothermal plate into an acoustic wave, which leads to dissipative thermal relaxation ( ). Therefore, we expect the acoustic wave to be attenuated. In the second case the acoustic power changes from negative to positive when the temperature gradient passes a critical value (marked as ∇Tcrit in the figure). More-over, the generated acoustic power is of the same order of magnitude as the viscous dissipation (O(pa)2). Therefore, we can expect acoustic oscillations to be sustained (at the critical transition point) or amplified.

To summarize, a uniform, temperature-dependent heat source can cause attenuation of an acoustic wave (consumption of acoustic power). More importantly, a nonuniform, temperature-dependent heat source can lead to generation of acoustic power, the magnitude of which depends on the sharpness of the gradient, and on the location of the heat source within the resonator.

### Non-Linear Radiation Model

Following the conclusions from section III, the motivation is established for seeking ways to create a strongly non-uniform, Temperature dependent heat source in an acoustic resonator, using radiation. We present here a simple configuration that enables such heat generation. A concentrated light beam illuminates the resonator from the axial direction, and is being absorbed by the fluid.

The beam enters the resonator through a window, but the illuminated section of the resonator is otherwise perfectly reflective, not allowing radiation to leave. In this section, we demonstrate that this configuration results in a non-uniform, temperature dependent heat source, and proceed to derive the equations governing the resultant thermoacoustic waves, which form the basis for the ensuing stability analysis.

We note that equation 14 demonstrates that any nonuniform profile of I will lead to a temperature dependent, non-uniform heat source, and can therefore potentially trigger an instability. We now proceed to manipulate the governing equations into a single ODE-a 1D wave equation. We follow and assume all variables are time-harmonic, of the form of equation (32). in which g1<<gm. In addition we assume Lresonator>>R, axis symmetry and negligible thermal conductivity (conductivity in the axial direction can be neglected due to a R<<λ, and in the radial direction it can be neglected if τα>>1). With these assumptions, equations (11-12) may be written as in equations (33-36). Here, u= [symbol] U·ex [symbol] is the cross-sectional-averaged velocity in the axial direction. Jn represents the nth order Bessel function of the first kind. In addition we have: um=0, ur=0, pm=pm(x), and q\dot{ } m=0. Equation 14 becomes equation (37). and we separate this equation into a mean and time dependent part, while neglecting terms of order O(T1)2 as equations (38) and (39), so that equation (35) is reduced to equation (40). Equations (33-34), along with equations (13) and (40) reduce to a single ordinary differential equation (41). This is a wave equation for the oscillating pressure p1. Given a known mean temperature profile, along with appropriate boundary conditions, this equation can be solved numerically.

### Limit Cycle Analysis

After an instability is triggered, an acoustic disturbance is expected to grow until the various dissipation mechanisms (mostly viscosity and thermal relaxation) dissipate the amount of power generated by the thermoacoustic amplification. In this section, we perform a limit-cycle analysis, based on equation 41, solving for the mean temperature, oscillating pressure and oscillating velocity fields under varying conditions corresponding with a specific heat input. The configuration considered is a 1D resonator with either a travelling wave or standing wave acoustic field. The limit cycle solution is then used to compute the predicted performance of radiation-driven thermoacoustic devices, in terms of efficiency and power density. To better account for losses commonly found in such systems, conduction within the surrounding solid wall of the resonator is added to the energy balance.

We begin by deriving the expression for total power transferred in the x direction in a radiation driven system as shown in equation (42). Where \hat{ }\dot{ }H is the total power transported in the irradiated segment, due to hydrodynamic dispersion, conduction in the solid part of the tube, and radiation within the media.

Here, α\hat{ }s and As are the solid thermal diffusivity and solid cross-sectional area, respectively. The first and second terms in equation 42 have been derived in the thermoacoustic literature and the last term has been derived in for radiative transfer in participating media with no flow.

The scaled total power and diffusivity are defined in equation (44) which is the equation for the scaled total power.

### Numerical Methodology

To perform the limit cycle analysis, equation 40 is substituted into equation 44, and manipulated into a differential equation for Tm. This equation is coupled with the wave equation (41), which are solved simultaneously for the distributions of p1, u1, and Tm. Both a travelling wave (L≈1, periodic boundary conditions on pressure, velocity and temperature) and standing wave (L≈½, no penetration boundary conditions on both sides) configurations are considered. In the non-irradiated segments, the standard acoustic propagation equations are solved, accounting for thermal relaxation, viscous dissipation and, where appropriate, turbulent losses. All the equations were solved using a variable order Runga-Kutta method with a Hybrid Powell shooting method, implemented with our in house solver—PC-TAS, a simulation software for 1D acoustics and thermoacoustic.

The working fluid considered is air at a pressure of 10 bar, however, in order to facilitate a simple variation of the radiation absorptivity k was assumed independent of the material properties, and was modified so as to probe the effect of to on system performance. We note that, in a physical realization of the system considered here, absorptivity could be modified by introduction of aerosols into the fluid, or by matching the radiation wavelength with fluid absorption lines. (i.e by irradiating a mixture of SF6 gas and air with a CO2 Laser). Either of these modifications might modify the other fluid properties (viscosity, density etc) considerably. We have ignored this effect mostly to keep the analysis simple and gain understating of the governing physics in such devices. The dimensional ambient temperature, T\hat{ }a, was kept constant at 400K, while the hot temperature TH was varied between 500-600K. Additional geometrical features, such as the location of the irradiated segment inside the resonator, the aspect ratio of the system, as well as the length of the Irradiated segment were roughly iterated via trial and error to improve system performance, but the system is not optimized.

In the travelling wave system, the irradiated segment was assumed wider than the nonirradiated segments, to increase the acoustic impedance p1/U1 of the sound wave in the irradiated area. This is common practice in travelling wave thermoacoustic, and indeed improved performance in the case presented here, as well. The total radiative power input into the system was set as a guess, targeting the prescribed hot temperature that corresponds with this amount of power.

The produced power is found by the calculating the difference between acoustic power on both sides of the irradiated segment. Efficiency was calculated by dividing the produced power by the total power, \dot{ }H, within the irradiated segment. Additional details about the Limit cycle analysis can be found in Appendix A.

### Results

Results show that the stability limit depends strongly on the radiation absorptivity K. In a Standing acoustic wave, the absorptivity should allow for comparable characteristic time scales for the oscillation, I/O, and heating \hat{ }ρcp/(4κσ\hat{ } T3). This is similar to the requirement in traditional thermoacoustic systems, for a comparable scale between the Oscillation time and the Thermal Diffusion characteristic time \hat{ }α/y2 0 (where \hat{ }α is the thermal diffusivity of the fluid and y0 is the pore size). We therefore define the parameter of equation (45) representing the ratio of the characteristic heating time and oscillation time in a radiation-driven system.

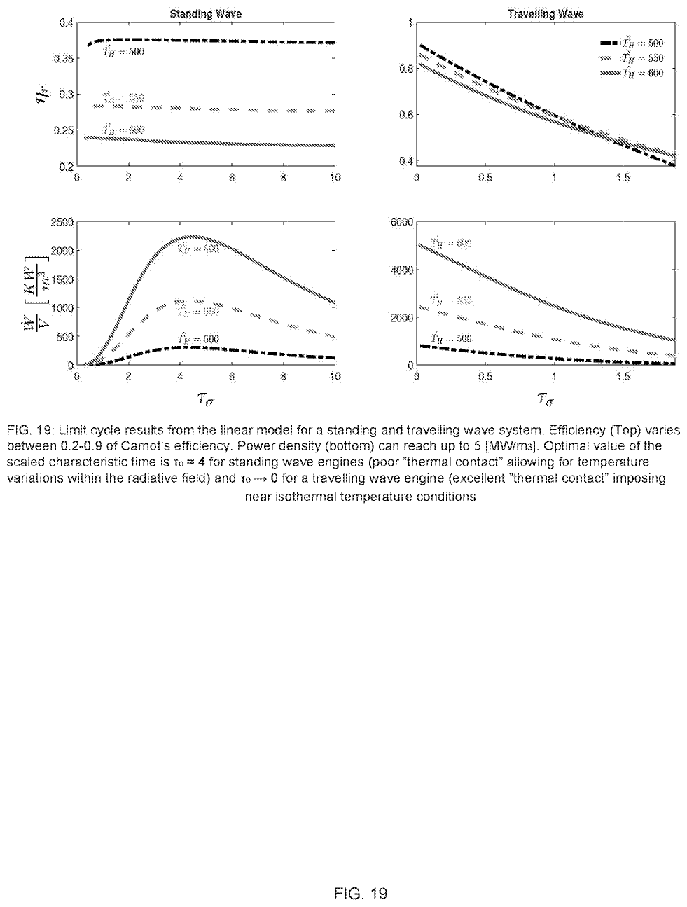

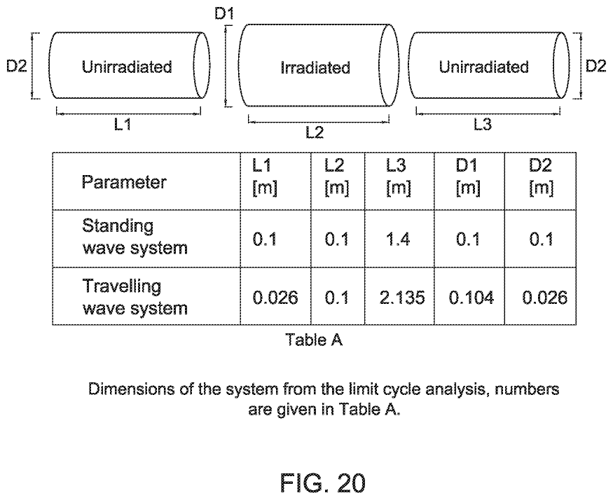

The calculated efficiency of heat-to-acoustic power conversion (relative to Carnot's limit−ηr=η/ηC) and energy density are shown in FIG. 19, for both standing wave and travelling wave configurations as a function of the hot temperature, T\hat{ }H, and of to. The results indicate high energy densities and excellent efficiencies for both standing and, particularly, travelling wave devices.

The predicted efficiency of both devices outperforms their ‘classical’, conduction-driven counterparts by more than a factor of 2 and the travelling wave device outperforms most existing heat engines. The dependence of the performance on τσ is substantial. As expected, standing wave devices achieve maximum performance at around τσ≈1 (4, to be exact, in the case considered), and travelling wave devices at around τσ→0. This is due to the nature of the thermodynamic cycles mimicked within the different acoustic fields, and is analogous to conduction driven devices, in which τα≈3 and τα→0 for standing and travelling wave devices, respectively (see).

While the model predictions are very encouraging, both in terms of illustrating the radiation-driven thermoacoustic instability, as well as the projected performance of devices implementing this mode of energy conversion, we must note several model limitations that could lead to performance over-prediction. First, the assumption of a grey medium is made, such that all spectral effects are neglected, as is scattering. Such effects could potentially hamper the performance of these devices, but they cannot be accounted for without knowledge of the actual absorbing media or radiation source.

However, in certain absorbing media (i.e very small dense suspended particles) spectral effects are negligible, and the scattering coefficient can be smaller than the absorption coefficient by several orders of magnitude. Second, no acoustic load was imposed on the system, and all of the generated acoustic power was dissipated by the resonator. In reality, the acoustic power will be consumed (e.g, by a thermoacoustic heat pump or acoustoelectric transducer), and resonator losses will be reduced but not eliminated. Separately accounting for the produced acoustic power within the irradiated segment and the power extracted from the system will reduce the calculated efficiency and power density by about 5-10%.

Further, the mechanisms of heat extraction from the ambient side were not addressed in this model. Any sort of heat exchanger will probably require a lower actual ambient temperature and in addition might add some vis cous attenuation of the acoustic wave. In addition, the assumption of 1D radiation propagation is only valid if the irradiated section is a perfect radiation cavity, i.e if the resonator walls are perfectly reflective. While near perfectly reflective materials exist, they are often difficult to implement in high temperature and pressure environments.

A reflectivity smaller than 80% (i.e polished aluminum) might cause substantial deviation from the model presented here. Finally, The effects of turbulence and heat conduction in the irradiated region as well as various non-linear mechanisms (e.g streaming) were not addressed by the model here, and may become important at high amplitudes.

We note also that neglecting thermal conductivity while keeping viscous terms is somewhat unjustified, seeing that the Prandtl number of most fluids is of order 1. This was done to simplify the equations considerably while keeping some dissipation mechanism in the equations. Both effects are taken into account in the unirradiated section, which consists of most of the length of the system.

According to an embodiment, there is provided a thermoacoustic engine that consists essentially of a heat exchanger and a resonator that is in communication with the heat exchanger, wherein the resonator includes s a media configured to absorb one or more electromagnetic signals. The thermoacoustic engine is configured to receive the one or more electromagnetic signals and generate acoustic power.

According to an embodiment, there is provided a method.

According to an embodiment, the method includes (i) receiving, by a thermoacoustic engine, one or more electromagnetic signals; and (ii) generating acoustic power, by the thermoacoustic engine and in response to the receiving of the electromagnetic signals. The thermoacoustic engine consists essentially of a heat exchanger and a resonator that is in communication with the heat exchanger, wherein the resonator includes s a media configured to absorb the one or more electromagnetic signals.

According to an embodiment, the one or more electromagnetic signals are one or more radiation signals.

According to an embodiment, the one or more electromagnetic signals are one or more electrical current signals.

According to an embodiment, each thermoacoustic engine lacks a stack.

According to an embodiment, each thermoacoustic engine lacks an additional heat exchanger.

According to an embodiment, the media is fluid.

According to an embodiment, the media is solid.

According to an embodiment, the heat exchanger is an ambient heat exchanger.

According to an embodiment, the method includes maintaining the heat exchanger at a temperature that is lower than a temperature of an illuminated portion of the resonator.

According to an embodiment, the media is gas, wherein gas located within an illuminated region of the resonator undergoes a thermoacoustic process.

According to an embodiment, the one or more electromagnetic signals are pulses of electromagnetic signals; and wherein the generating of the acoustic includes s generating acoustic waves.

According to an embodiment, the method further includes receiving, by an additional thermoacoustic engine, one or more additional electromagnetic signals; and generating acoustic power, by the additional thermoacoustic engine and in response to the receiving of the one or more additional electromagnetic signals. The additional thermoacoustic engine consists essentially of an additional heat exchanger and an additional resonator that is in communication with the additional heat exchanger, wherein the additional resonator includes s a media configured to absorb one or more additional electromagnetic signals that are received by the additional thermoacoustic engine.

According to an embodiment, the method further includes receiving, by additional thermoacoustic engines, additional electromagnetic signals; and generating acoustic power, by the additional thermoacoustic engines and in response to the receiving of the additional electromagnetic signals. Each one of the additional thermoacoustic engines consists essentially of an additional heat exchanger and an additional resonator that is in communication with the additional heat exchanger. The additional resonator includes a media configured to absorb one or more additional electromagnetic signals that are received by the additional thermoacoustic engine.

According to an embodiment, the method includes distributing a plurality of electromagnetic signals between a plurality of thermoacoustic engines, wherein the plurality of thermoacoustic engines includes s the additional thermoacoustic engines and the thermoacoustic engine, wherein the plurality of electromagnetic signals includes s the one or more electromagnetic signals and the additional electromagnetic signals.

According to an embodiment, the plurality of electromagnetic signals are a plurality of radiation signals.

According to an embodiment, the plurality of electromagnetic signals are a plurality of pulses of radiation.

According to an embodiment, the distributing is executed using a wavelength based distribution element.

According to an embodiment, the distributing is executed using a polarization based distribution element.

According to an embodiment, there is provided an energy providing unit that includes one or more thermoacoustic engines.

Each thermoacoustic engine of the one or more thermoacoustic engines consists essentially of a heat exchanger and a resonator that is in communication with the heat exchanger, wherein the resonator includes a media configured to absorb one or more electromagnetic signals. The thermoacoustic engine is configured to receive the one or more electromagnetic signals and generate acoustic power.

According to an embodiment, the electromagnetic signals are radiation signals and wherein the energy providing unit consisting essentially of the one or more thermoacoustic engines and a radiation illuminating unit configured to generate the radiation signals.

According to an embodiment, the one or more thermoacoustic engines are multiple thermoacoustic engines.

According to an embodiment, the radiation illumination unit includes a radiation distributing unit for distributing the radiation signals between the multiple thermoacoustic engines.

According to an embodiment, the radiation signals are pulses of radiation and wherein the distributing unit is configured to distribute the pulses of the radiation between the multiple thermoacoustic engines.

According to an embodiment, the radiation distributing unit includes s a wavelength based distribution element.

According to an embodiment, the radiation distributing unit includes s a polarization based distribution element.

### CONCLUSIONS

In this work, a radiation-driven mode of thermoacoustic instability was examined theoretically. Employing a simplified model, it was demonstrated how any nonuniform, temperature-dependent heat source can cause generation of acoustic power in a standing acoustic wave.

Such sources include, but are not limited to, irradiation of a medium.

Indeed, the framework presented here might be used to investigate any kind of volumetric heat source, including combustion, nuclear or chemical reactions, or the spaced-averaged effect of a “classical” conduction driven thermoacoustic system.

A more elaborate model was then used to derive a wave equation, describing the acoustic field. A stability analysis was performed, predicting the threshold for onset of oscillations and its dependence on the acoustic configuration.

A limit cycle analysis demonstrated the potentially superior performance of radiation-driven thermoacoustic devices over existing ones. Results indicated that reasonable stability limits depend heavily on matching the characteristic heating time with the acoustic oscillation period. Such matching requires highly absorbing media, for example, atmospheric air at a mean temperature of 700K will require κ≈10,000 for τσ=1. Therefore, experimental manifestation of this phenomenon in future research will require methods for increasing the working media's absorptivity, with options including particulate aerosols, super-critical fluids and compressible liquids

While the foregoing written description of the invention enables one of ordinary skill to make and use what is considered presently to be the best mode thereof, those of ordinary skill will understand and appreciate the existence of variations, combinations, and equivalents of the specific embodiment, method, and examples herein. The invention should therefore not be limited by the above described embodiment, method, and examples, but by all embodiments and methods within the scope and spirit of the invention as claimed.

In the foregoing specification, the invention has been described with reference to specific examples of embodiments of the invention. It will, however, be evident that various modifications and changes may be made therein without departing from the broader spirit and scope of the invention as set forth in the appended claims.

Other modifications, variations and alternatives are also possible. The specifications and drawings are, accordingly, to be regarded in an illustrative rather than in a restrictive sense.

In the claims, any reference signs placed between parentheses shall not be construed as limiting the claim. The word ‘comprising’ does not exclude the presence of other elements or steps then those listed in a claim. Furthermore, the terms “a” or “an,” as used herein, are defined as one or more than one. Also, the use of introductory phrases such as “at least one” and “one or more” in the claims should not be construed to imply that the introduction of another claim element by the indefinite articles “a” or “an” limits any particular claim containing such introduced claim element to inventions containing only one such element, even when the same claim includes the introductory phrases “one or more” or “at least one” and indefinite articles such as “a” or “an.” The same holds true for the use of definite articles. Unless stated otherwise, terms such as “first” and “second” are used to arbitrarily distinguish between the elements such terms describe. Thus, these terms are not necessarily intended to indicate temporal or other prioritization of such elements. The mere fact that certain measures are recited in mutually different claims does not indicate that a combination of these measures cannot be used to advantage.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

It is appreciated that various features of the embodiments of the disclosure which are, for clarity, described in the contexts of separate embodiments may also be provided in combination in a single embodiment. Conversely, various features of the embodiments of the disclosure which are, for brevity, described in the context of a single embodiment may also be provided separately or in any suitable sub-combination.

It will be appreciated by persons skilled in the art that the embodiments of the disclosure are not limited by what has been particularly shown and described hereinabove. Rather the scope of the embodiments of the disclosure is defined by the appended claims and equivalents thereof.

1. A thermoacoustic engine that consists essentially of:
a heat exchanger; and
a resonator that is in communication with the heat exchanger, wherein the resonator comprises a media configured to absorb one or more electromagnetic signals; and
wherein the thermoacoustic engine is configured to receive the one or more electromagnetic signals and generate acoustic power.

2. (canceled)

3. The thermoacoustic engine according to claim 1, wherein the one or more electromagnetic signals are one or more electrical current signals.

4. The thermoacoustic engine according to claim 1, wherein each thermoacoustic engine lacks a stack.

5. The thermoacoustic engine according to claim 1, wherein each thermoacoustic engine lacks an additional heat exchanger.

6. The thermoacoustic engine according to claim 1, wherein the media is fluid.

7. The thermoacoustic engine according to claim 1, wherein the media is solid.

8. The thermoacoustic engine according to claim 1, wherein the heat exchanger is an ambient heat exchanger.

9. The thermoacoustic engine according to claim 1, wherein the heat exchanger is configured to be maintained at a temperature that is lower than a temperature of an illuminated portion of the resonator.

10. The thermoacoustic engine according to claim 1, wherein the media is gas, wherein gas located within an illuminated region of the resonator undergoes a thermoacoustic process.

11. The thermoacoustic engine according to claim 1, wherein the thermoacoustic engine is configured to generate acoustic waves when exposed to pulses of electromagnetic signals.

12. A method, comprising:
receiving, by a thermoacoustic engine, one or more electromagnetic signals; and
generating acoustic power, by the thermoacoustic engine and in response to the receiving of the electromagnetic signals; wherein the thermoacoustic engine consists essentially of a heat exchanger and a resonator that is in communication with the heat exchanger, wherein the resonator comprises a media configured to absorb the one or more electromagnetic signals.

13. (canceled)

14. (canceled)

15. (canceled)

16. (canceled)

17. (canceled)

18. (canceled)

19. (canceled)

20. (canceled)

21. (canceled)

22. (canceled)

23. The method according to claim 1, further comprising:
receiving, by an additional thermoacoustic engine, one or more additional electromagnetic signals; and
generating acoustic power, by the additional thermoacoustic engine and in response to the receiving of the one or more additional electromagnetic signals;
wherein the additional thermoacoustic engine consists essentially of an additional heat exchanger and an additional resonator that is in communication with the additional heat exchanger, wherein the additional resonator comprises a media configured to absorb one or more additional electromagnetic signals that are received by the additional thermoacoustic engine.

24. The method according to claim 1, further comprising:
receiving, by additional thermoacoustic engines, additional electromagnetic signals; and
generating acoustic power, by the additional thermoacoustic engines and in response to the receiving of the additional electromagnetic signals;
wherein each one of the additional thermoacoustic engines consists essentially of an additional heat exchanger and an additional resonator that is in communication with the additional heat exchanger, wherein the additional resonator comprises a media configured to absorb one or more additional electromagnetic signals that are received by the additional thermoacoustic engine.

25. The method according to claim 23, comprising distributing a plurality of electromagnetic signals between a plurality of thermoacoustic engines, wherein the plurality of thermoacoustic engines comprises the additional thermoacoustic engines and the thermoacoustic engine, wherein the plurality of electromagnetic signals comprises the one or more electromagnetic signals and the additional electromagnetic signals.

26. The method according to claim 25, wherein the plurality of electromagnetic signals are a plurality of radiation signals.

27. The method according to claim 25, wherein the plurality of electromagnetic signals are a plurality of pulses of radiation.

28. The method according to claim 25 wherein the distributing is executed using a wavelength based distribution element.

29. The method according to claim 25 wherein the distributing is executed using a polarization based distribution element.

30. An energy providing unit, comprising:
one or more thermoacoustic engines;
wherein each thermoacoustic engine of the one or more thermoacoustic engines consists essentially of a heat exchanger and a resonator that is in communication with the heat exchanger, wherein the resonator comprises a media configured to absorb one or more electromagnetic signals; and wherein the thermoacoustic engine is configured to receive the one or more electromagnetic signals and generate acoustic power.

31. (canceled)

32. (canceled)

33. (canceled)

34. (canceled)

35. (canceled)

36. (canceled)

37. (canceled)
